(19) United States
(12) Patent Application Publication
(10) Pub. No.: US 2009/0264714 A1
(43) Pub. Date: Oct. 22, 2009

(54) NON-INVASIVE VITAL SIGN MONITORING METHOD, APPARATUS AND SYSTEM

(76) Inventor: Chang-An Chou, Taipei (TW)

Correspondence Address: Chang-An Chou, 3F, No. 100, Sec. 3. Mingsheng E. Rd., Taipei, 105, TW

(21) Appl. No.: 12/300,128

(22) PCT Filed: May 18, 2007
(86) PCT No.: PCT/CN07/01619

§ 371 Date: Nov. 10, 2008

(30) Foreign Application Priority Data
May 18, 2006 (CN) 200610082707.7

(51) Int. Cl. A61B 5/00

(52) U.S. Cl. 600/301

(57) ABSTRACT

A non-invasive vital sign monitoring apparatus includes a detecting device (108) attached to a first user though an attaching element (110), and a receiving device (102) held by a second user for wirelessly receiving the signals and messages from the detecting device (108). The detecting device (108) includes a detecting unit (116-128) for gathering a physiological signal from the first user, a sensing mechanism (114) mounted on the attaching element (110), having a combined state and a released state for corresponding to the attaching relationship thereof with the attaching element (110), and a control unit (112) for controlling the operation of the detecting device (108), wherein the control unit (112) triggers a warning message when the gathered physiological signal does not conform to a preset physiological condition, the control unit (112) triggers a notification of released state when the sensing mechanism (114) is under the released state, and in accordance with the states of the sensing mechanism (114), the control unit (112) drives the detecting device (108) to enter different operation modes. Furthermore, when receiving the warning message, the receiving device (102) sends out a warning notice to show the second user a disconformity between the gathered physiological signal and the preset physiological condition, and when receiving the notification of released state, the receiving device (102) sends out a release notice to the second user.

### FIELD OF THE INVENTION

The present invention is related to a vital sign monitoring method, apparatus, and system, and more particularly, to a non-invasive vital sign monitoring method, apparatus and system for correctly providing an instant warning.

### BACKGROUND OF THE INVENTION

The nursing demands in hospitals and home are gradually increased since the aging problem has become a serious issue in the modern society. However, nursing care requires the supports of manpower and financial resources, especially for those requiring long-term or full time care, so that there have many ideas being disclosed for solving this problem.

Among the solutions, electronic equipments, which already influence our life deeply, are introduced into the medical field for solving the problem described above, wherein the monitoring device and communication technology are employed to replace the human company, and thus, the goal of taking care of multiple patients simultaneously by one person can be easily achieved.

Traditionally, the monitoring is focused on transmitting the sound or picture of the patient to the carer at other place, for example, utilizing a microphone or camera. The most popular application is to take care of baby(s). The parents or the babysitter don't have to accompany aside the baby and can work, for example, in another room, with the receiver, such as, a speaker or a screen, so that the full attention to the baby is only required as the receiver shows any abnormality.

Another kind of application is to take care of adults. For example, in the sickroom, there always has an emergency button for notifying the nursing department the emergency situation occurred therein, so that the medical personnel can have an immediate response.

As described, the above-described applications are based on a passive notification, that is, if the baby is smothered without crying, then it will be difficult for the carer to notice this lethal condition, or if the patient stays in the sickroom by himself, then he might not have the capability to press the emergency button for asking rescue in some circumstances. Therefore, improvements are necessary.

For solving the problem, monitoring physiological parameters becomes the major way to realize testee's physical conditions. Generally, physiological parameter monitoring employs many sensors to attach to the monitored person for gathering physiological parameters, which are then transmitted back to a remote receiver, so that when abnormal change in physiological parameters occurs, the monitor can act properly. Furthermore, a warning system might also be included for notifying the monitor the abnormal situation.

However, one drawback of this kind of device is the complicated sensor arrangement, which obviously is a burden on the user and which might not be easily completed by the user himself. Besides, since the warning system is always set to be triggered only when the selected physiological parameter is beyond a normal range, this is still a passive notifying for the monitor, but oppositely, a real time transmission has to consume a large amount of electricity, which is undesired in most applications, above all, for the battery operated devices. Therefore, it is difficult to complete in both respects. In addition, an accident situation that the sensor comes off the human body will also trigger the warning system, so that it is hard for the monitor to differentiate this from the real danger happened on the user since the physiological parameter also shows abnormality in this situation.

Consequently, the object of the present invention is to provide a non-invasive vital sign monitoring apparatus with a high-efficient power utilization, so as to actively provide accurate, instant physiological information and status related to the person to be monitored.

Another object of the present invention is to a non-invasive vital sign monitoring apparatus employing a sensing mechanism, which can discover the coming off of the sensor, so as to reduce the incorrect warning.

Another further object of the present invention is to provide a non-invasive vital sign monitoring apparatus, in which the monitor at the receiving end can actively request the detecting end to transmit back requested information, so that the monitor can be easily aware of the real physiological situation of the user without wasting power.

### SUMMARY OF THE INVENTION

A non-invasive vital sign monitoring apparatus including a detecting device, attached to a first user through an attaching element and a receiving device, held by a second user, for wirelessly receiving the signals and messages from the detecting device is disclosed. The detecting device includes a detecting unit, for gathering a physiological signal from the first user, a sensing mechanism, mounted on the attaching element, having a combined state and a released state for corresponding to the attaching relationship thereof with the attaching element, and a control unit, for controlling the operation of the detecting device, wherein the control unit triggers a warning message when the gathered physiological signal does not conform to a preset physiological condition, the control unit triggers a notification of released state when the sensing mechanism is under the released state, and in accordance with the states of the sensing mechanism, the control unit drives the detecting device to enter different operation modes. Furthermore, when receiving the warning message, the receiving device sends out a warning notice to show the second user a disconformity between the gathered physiological signal and the preset physiological condition, and when receiving the notification of released state, the receiving device sends out a release notice to the second user.

Preferably, the physiological signal is a heartbeat signal and/or a pulse signal, and the detecting unit is an optical detecting unit comprising an optical emitting element and an optical receiving element, wherein the quantity of the optical emitting element and the optical receiving element are both changeable, and the optical detecting unit is attached to the first user by clipping, surrounding or adhering, and at finger, ear, or forehead.

More advantageously, the apparatus can further include a piezo-type detecting element, attached to the first user, for gathering another kind of physiological signal from the first user, wherein the piezo-type detecting element is used for detecting signals generated by skin surface vibration and/or skin expansion/contraction, and the piezo-type detecting element is attached to the first user by adhering, surrounding or binding, and at the chest or the neck.

More advantageously, the apparatus can further include a non-invasive physiological electrode, attached to the first user, for gathering another kind of physiological signal from the first user.

More advantageously, the apparatus can further include a movement sensing element, wherein the movement sensing element is located inside the detecting device, and the signals gathered by the movement sensing element are used to be a judging reference of other signals.

More advantageously, the apparatus can further include a sound sensing element, for providing a sound output by the first user or by the environment around the first user to the second user.

More advantageously, the apparatus can further include a temperature sensing element, for providing a body temperature information and/or an environment temperature information.

Furthermore, the combined state of the sensing mechanism may trigger the control unit to send out a notification of combined state, and the sensing mechanism is located between the attaching element and the detecting device, and/or between the attaching element and the first user.

Moreover, the detecting device may further include a button, for being used by the first user to trigger a mayday message, and then the receiving device, after receiving the mayday message, sends out a mayday notice and simultaneously sends out a rescue-requesting message to an emergency system.

Preferably, the notices sent by the receiving device are implemented as sound, light, image and/or vibration.

More advantageously, the signals and messages sent by the detecting device are received by multiple receiving devices, and/or the receiving device can receive signals and messages from multiple detecting devices.

In another aspect of the present invention, a non-invasive vital sign monitoring apparatus is provided. The apparatus includes a detecting device, attached to a first user through an attaching element for gathering physiological signals from the first user, including a sensing mechanism, mounted on the attaching element, having a combined state and a released state for corresponding to an attaching relationship thereof with the attaching element, wherein according to the states of the sensing mechanism, the detecting device are changed to enter different operation modes; and a receiving device, held by a second user, including an operation interface, which is utilized by the second user to send out a requesting message for requesting the detecting device to transmit back a particular physiological signal.

Preferably, the different operation modes have different power supplying conditions.

In further another aspect of the present invention, a non-invasive method for monitoring physiological signals includes steps of a) providing a detecting device with an attaching element and a receiving device; b) providing a sensing mechanism, having a combined state and a release state, on the detecting device; c) attaching the detecting device on a user through the attaching element; d) gathering a physiological signal; e) monitoring the states of the sensing mechanism; f) sending out a notification of released state to the receiving device as the detecting device detects the released state; and g) sending out a release notice as the receiving device receives the notification of released state for showing the release state.

More advantageously, the method may further include the steps of h) as the combined state is detected, sending out a notification of combined state by the detecting device to the receiving device; and i) sending out a combination notice by the receiving device after receiving the notification of combined state.

In still another aspect of the present invention, a non-invasive method for monitoring physiological signals includes steps of a) providing a detecting device with an attaching element and a receiving device; b) providing an operation interface on the receiving device; c) attaching the detecting device on a user through the attaching element; d) gathering a physiological signal; e) sending out a requesting message through the operation interface, so as to request the detecting device to transmit a particular physiological signal; and f) transmitting the requested physiological signal to the receiving device after the detecting device receives the requesting message.

In another further aspect of the present invention, a non-invasive vital sign monitoring system includes at least a non-invasive vital sign monitoring apparatus and at least an administration console. The monitoring apparatus includes a detecting device, attached to a user through an attaching element, having a detecting unit, for gathering a physiological signal from the first user, a sensing mechanism, mounted on the attaching element, having a combined state and a released state for corresponding to an attaching relationship thereof with the attaching element, and a control unit, for controlling the operation of the detecting device, wherein the control unit triggers a warning message when the gathered physiological signal does not conform to a preset physiological condition, the control unit triggers a notification of released state when the sensing mechanism is under the released state, and the control unit drives the detecting device to enter different operation modes in accordance with the states of the sensing mechanism; and a receiving device for wirelessly receiving the physiological signals and messages from the detecting device. Furthermore, when receiving the warning message, the receiving device sends out a warning notice to the administration console for showing a disconformity between the gathered signals and the preset physiological condition, and when receiving the notification of released state, the receiving device sends out a release notice to the administration console for showing the attaching situation of the detecting device.

Preferably, the vital sign monitoring apparatus is connected with the administration console through a network system, which is a wired or wireless network system.

More advantageously, the administration console may send out a requesting message for requesting a physiological signal from the detecting device, and the requesting message is transmitted to the receiving device and then to the detecting device, and the detecting device transmits back the physiological signal after receiving the requesting message.

### BRIEF DESCRIPTION OF THE DRAWINGS

A more detailed understanding of the invention may be had from the following description of a preferred embodiment, given by way of example, and to be understood in conjunction with the accompanying drawings, wherein:

FIG. 1 is a schematic view showing a non-invasive vital sign monitoring apparatus according to the present invention;

FIGS. 2A˜2F show various implementation types of the detecting device and the attaching element in the non-invasive vital sign monitoring apparatus according to the present invention;

FIG. 3 is a flow chart showing the warning process of the non-invasive vital sign monitoring apparatus according to the present invention;

FIGS. 4A˜4E show various possible embodiments of the sensing mechanism according to the present invention;

FIGS. 5A˜5B are flow charts showing the notification process of the sensing mechanism according to the present invention;

FIG. 6 is a flow chart showing the process that the receiving device actively requests the information transmission according to the present invention;

FIG. 7 is a flow chart showing the mayday process according to the present invention;

FIGS. 8A˜8C show the possible examples of the receiving device according to the present invention;

FIG. 9 is a schematic view showing one kind of relationship between the detecting devices and the receiving device;

FIG. 10 is a schematic view showing another kind of relationship between the detecting device and the receiving devices; and

FIG. 11 is a schematic view showing a non-invasive vital sign monitoring system according to the present invention.

### DETAILED DESCRIPTION OF THE PREFERRED EMBODIMENTS

The present invention provides an apparatus for non-invasively and instantly monitoring vital sign which provides not only a warning notice to notify the occurrence of abnormal vital sign, but also a notice representing the coming off of the detecting device, so as to confirm the accuracy of the warning notice. Besides, through the receiving device, the monitor can actively request the detecting device to transmit back information related thereto so as to achieve a completed and effective two-way communication.

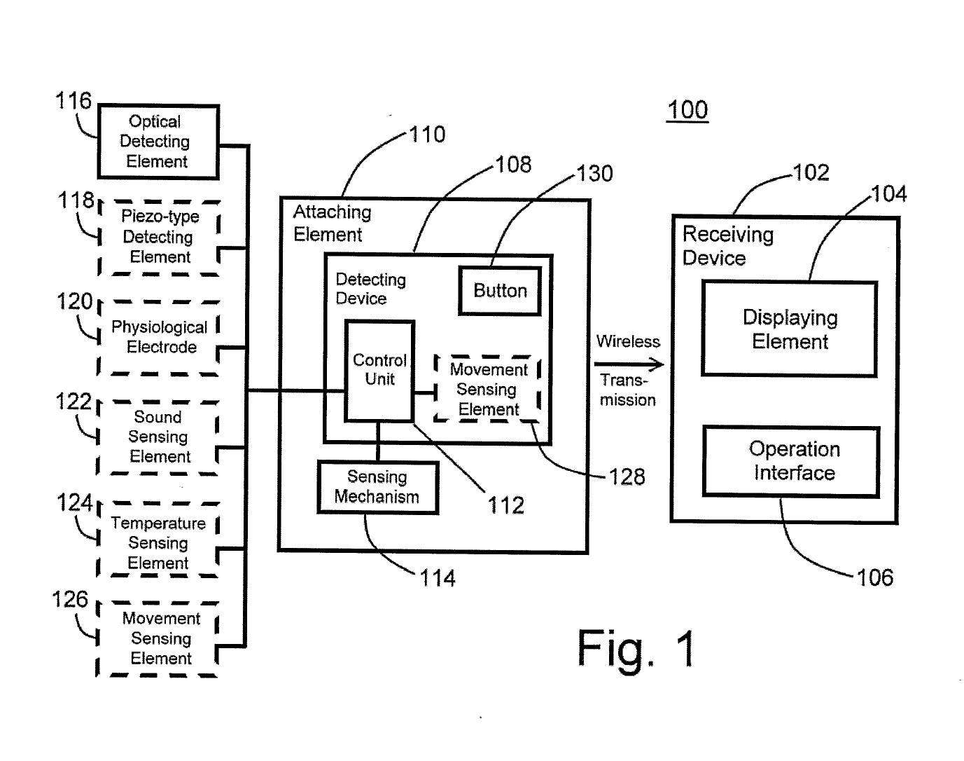

Please refer to FIG. 1, which is a schematic view showing the non-invasive vital sign monitoring apparatus according to the present invention. As shown, the non-invasive vital sign monitoring apparatus 100 includes two major parts, one is a detecting device 108 attached to a user and the other is a receiving device 102 monitored by a monitor, and the detecting device 108 is attached to the user through an attaching element 110. The detecting device 108 includes at least a physiological signal detecting element 116˜128, a sensing mechanism 114 and a control unit 112, and the receiving device 102 includes a displaying element 104 and an operation interface 106.

According to the purposes of the present invention, the detecting device and the receiving device are respectively arranged on the user and on the monitor who does not stay aside the user, so that the communication therebetween must be implemented as wireless.

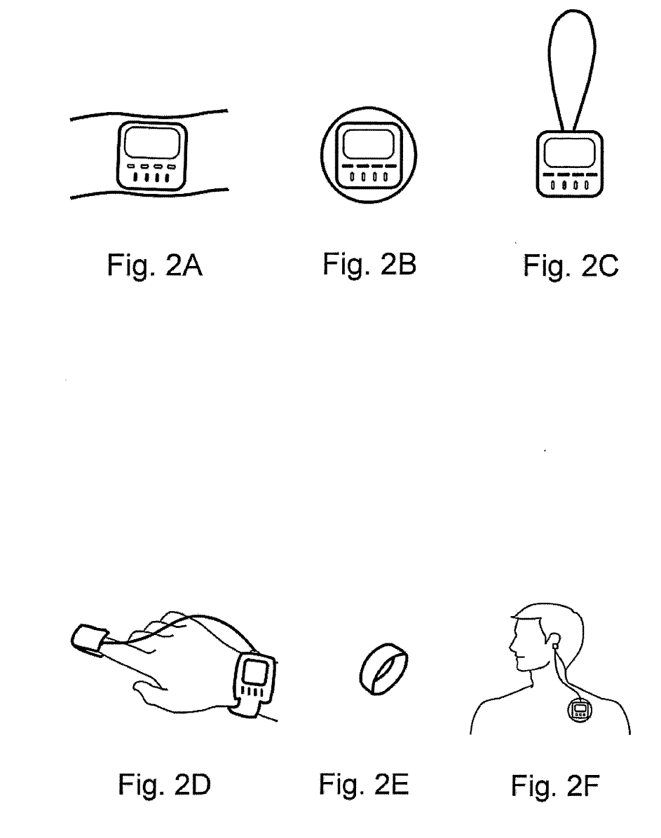

Furthermore, since the detecting device is attached to the user through the attaching element, for achieving this attachment and also for reducing the user's burden, the detecting device is implemented to have a small volume and a light weight. As to the attaching element, according to different attaching positions, it can be implemented into different types, for example, a belt (FIG. 2A), a patch (FIG. 2B) or a handing band (FIG. 2C). However, the type thereof is not limited, and the major principle is not to increase the burden on the user.

The detecting element is employed to gather physiological signals from the user. In the present invention, the physiological signal can be a heartbeat because it perhaps is the most representative and most easily obtained physiological signal for a living body.

The reasons for choosing heartbeat as the physiological signal to be monitored are as followed. Generally, no matter which kind of physical situation happens, the heartbeat is directly influenced. Moreover, heartbeat is an easily obtained physiological signal, which means the user can easily complete the arrangement of heartbeat detecting element without any professional training. Besides, there are many sources for obtaining heartbeat, such as, the contraction of blood vessel, the heart sounds from heart beating, and the skin vibration from pulse etc. And, there is almost no limitation for positioning the heartbeat detecting element.

Except the description above, taking heartbeat as the physiological signal for monitoring vital sign is advantageous that heartbeat is a stable physiological signal which is not easily influenced by external factors, such as, body and limb movement.

Most importantly, heartbeat is the best value representing the stability of human life. Particularly, when judging the aged people with chronic disease, the patients in danger of some fulminating diseases, or the speechless babies, how to confirm the physical condition thereof is under a stable state is significantly important.

Therefore, heartbeat/pulse signal is actually a physiological signal which can accurately represent the vital sign of a human body without too many limitations.

As to the detecting element to be used, there are many choices;

### (1) Optical Detecting Unit

The optical detecting unit is a combination of optical emitting element and optical receiving element. When heart beats, the blood vessel connecting to the heart will relatively have a contraction, so that the absorption variation of the emitted light which passes through the blood vessel can represent the contraction frequency of the blood vessel, so as to realize the heartbeat frequency.

### (2) Pizeo-Type Detecting Element

The pizeo-type detecting element senses the vibration at the skin surface caused by heart beating and/or blood vessel contraction, and the vibration changing can be transformed into current variation for calculating heartbeat/pulse alteration.

### (3) Physiological Electrodes

The ECG (electrocardiograph) electrodes can be employed. Here, since it only needs to know the change of heartbeat/pulse, the arranged positions of the ECG electrodes are not as critical as the ECG monitoring, so that there is no difficulty for the user in arranging.

Of course, except the heartbeat/pulse, other physiological parameters also can be used for monitoring and/or assisting the judgment, so that a better result can be obtained through a cross reference therebtween. One choice is the movement sensing element, for providing the moving information of the user. The movement sensing element can be located in the detecting device 108 (as indicated by 128 shown in FIG. 1), or can be combined with other detecting element (as indicated by 126 shown in FIG. 1). Alternatively, a GPS can be utilized for providing the location of the user. Another choice is the temperature sensing element 124, which is used for providing body temperature or/and environmental temperature information for judge assistance. Of course, these are only for illustration, and not for limitation.

FIGS. 2A˜2F show various implementation types for the detecting device and the attaching element in the non-invasive vital sign monitoring apparatus according to the present invention, wherein in accordance with different kinds of physiological signals and different detecting elements, and simultaneously considering the comfort, convenience and mobility of the user, the combination of the detecting device and the attaching element can have many choices. For example, it can be implemented as a watch type detecting device with a clipping type detecting element (FIG. 2D), a ring type combined detecting element and device (FIG. 2E), or a patch type detecting device near the ear with an ear-clipping detecting element (FIG. 2F). Moreover, if the arranging position of the detecting device is not limited, then the attaching element can be implemented into a common type, such as, a belt for head, chest, arm or wrist (FIG. 2A), an adhering patch for almost whole skin surface, and a hanging band (FIG. 2C). Alternatively, the detecting elements in FIGS. 2A and 2B can be directly integrated with the belt or patch, or FIGS. 2B and 2C can be combined together to become another kind of implementation. Therefore, all these described examples are only depicted for illustration and not for limitation.

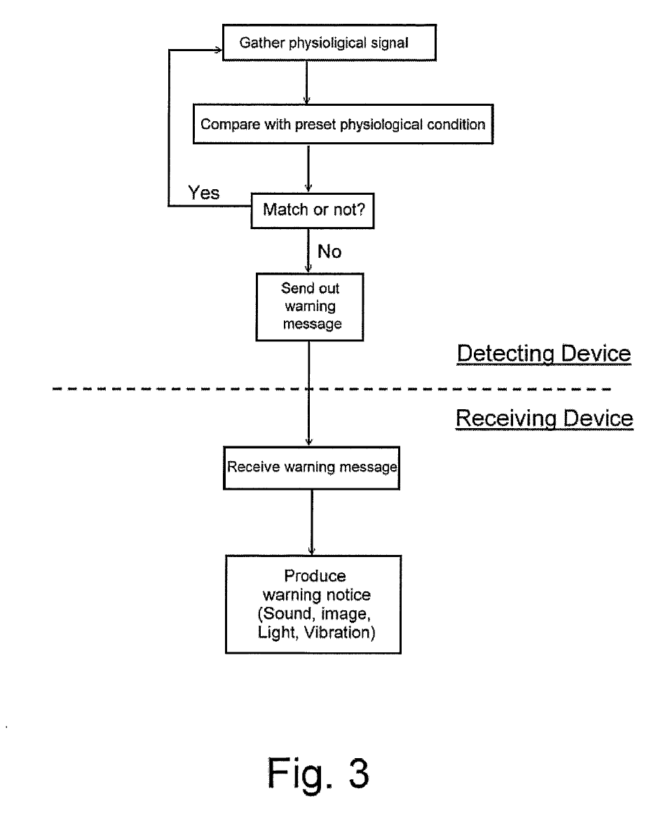

As to the control unit in the detecting device, it is used to control the operation of the detecting device. Therefore, the warning function, as described above, provided by the non-invasive vital sign monitoring apparatus according to the present invention is controlled by the control unit. The process for generating warning message is shown in FIG. 3. Firstly, after gathering the physiological signals, the gathered signals are compared to a preset physiological condition. Then, if the gathered signals do not match to the preset condition, the control unit immediately sends out a warning message to the receiving device, and after receiving the warning message, the receiving device produce a warning notice for notifying the monitor. Here, the notice can be implemented into various types, such as, sound, light, vibration or image.

Since the detecting device and the receiving device are wirelessly communicated with each other, battery consumption becomes a serious problem, especially the detecting device is implemented to be portable. Therefore, if it is implemented to be a real time wireless transmission, then the principles of weightless and volume reduction must conflict with the long-term power supply. For example, for keeping the device light and small, the battery lasting time might lower to less than 24 hours, or for providing sufficient power, it is unavoidable that the volume and weight will be sacrificed. Accordingly, the present invention provides different operation modes with different power consuming conditions for conforming to different operation demands.

First, in regard to signal gathering, the control unit is designed to proceed an intermittent gathering, and during the non-detecting period, the detecting device will get into a power-saving mode, such as a sleep mode, so as to reduce power consumption.

As to data transmission, according to the present invention, it also has different modes, for example, but not limited, a common mode and an event trigger mode.

In common mode, the gathered physiological signals are intermittently transmitted to the receiving device. Here, the transmission interval also can have different choices, for example, at the beginning of measurement, the transmission interval can have a more concentrated frequency, and then, as the signals become more stable, the transmission interval can be extended. Except the physiological signal transmission, between the detecting device and the receiving device, a transmission of a connection confirmation message also has to be performed constantly, so as to verify the wireless communication therebetween. Accordingly, the physiological signals can be transmitted together with the connection confirmation message, for example, every time, every two times, or every three times the connection confirmation message is transmitted. It should be noticed that if the physiological signal is discovered to not conform to the preset condition, the detecting device will immediately be triggered to send out the warning message to the receiving device, no matter whether the detecting device is processing the physiological signal transmission.

In the event trigger mode, the detecting device also gathers the physiological signals intermittently through the detecting element. But, differently, in this mode, the physiological signals are not transmitted periodically, that means, the detected physiological information is kept at the detecting end and not transmitted to the receiving end. However, if any abnormality of the physiological information occurs, the detecting device will immediately be triggered to send out the warning message, so as to produce the warning notice. Therefore, in this mode, the detecting device only delivers the warning message and the connection confirmation message, so that the power consumption is reduced to the lowest level. Here, it is important that the warning and rescuing functions are still maintained.

In the prior arts, it is always that when the gathered physiological signals do not confirm to the preset physiological condition, a warning message is sent out by the detecting device, or the detecting device itself produces a warning notice. However, the coming off of the detecting device is not considered. The problem is that the detecting device might come off the body of the user, especially when the user is sleeping or unconscious, so that, at this time, the gathered physiological will not conform to the present physiological condition, and definitely, the warning message will be triggered by the detecting device. But, unfortunately, the monitor, such as, the carer, or the medical personnel, can not differentiate the difference between the real abnormality and the false warning, and thus, it is for sure that the monitor must immediately reach to the monitored person for safety. Therefore, the manpower is wasted in this situation. Besides, if the warning is directly connected to the emergency medical service system, then it will cause an even more manpower wasting.

Hence, except instantaneity and power-saving, information accuracy also plays an important role in vital sign monitoring. Accordingly, the present invention proposes a design for preventing the false warning, and for achieving thereof, the detecting device further includes a sensing mechanism 114, as shown in FIG. 1. Since the detecting device is attached to the user through the attaching element, the coming off is decided by if the attaching element is departed from the user and/or if the detecting device is departed from the attaching element. Therefore, the sensing mechanism 114 is mounted on the attaching element 110 and is located between the attaching element and the detecting device and/or between the attaching element and the user.

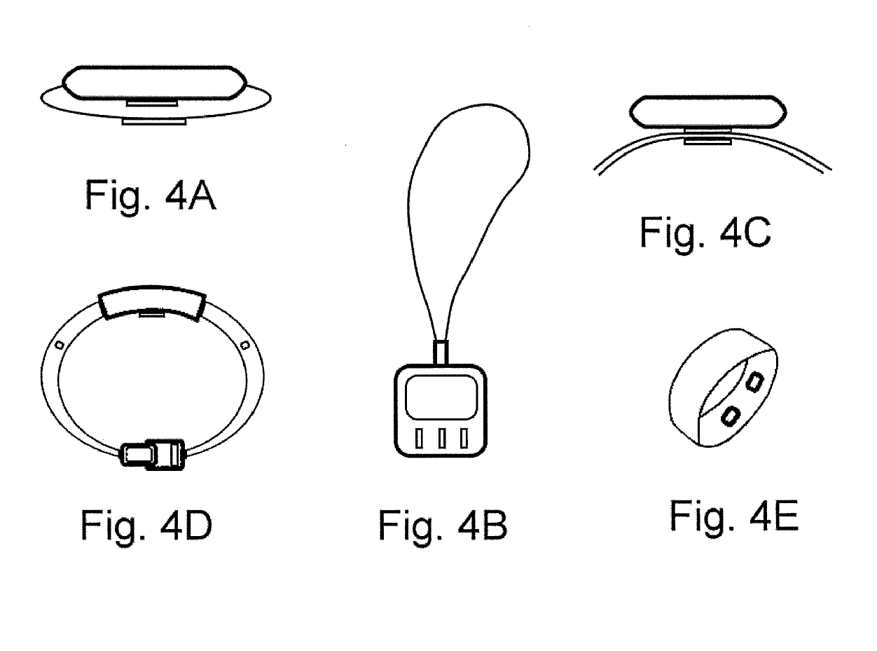

FIGS. 4A˜4E show some possible embodiments of the sensing mechanism 114. According to different attaching elements, the sensing mechanism 114 is implemented into different types and to locate at different positions. For example, if the attaching element is an adhering patch, as shown in FIG. 4A, the sensing mechanism 114 might be implemented to locate between the attaching element and the user and/or between the attaching element and the detecting device, so that once the attaching element departs from the user and/or the detecting device separates from the attaching element, the sensing mechanism 114 can sense the separation at the first time, so as to instantly responding the released situation. In this embodiment, preferably, the sensing mechanism 114 can be implemented to be a pressure sensing mechanism. Then, if the attaching element is implemented as a hanging band, as shown in FIG. 4B, the sensing mechanism 114 can be designed to locate at the combination point between the hanging band and the device and/or inside the hanging band (not shown) for sensing the broken of the band or sensing the separation of the band from the user. Besides, if the attaching element is implemented to be a winding belt, such as, a binding belt, a watch-type belt or a ring-type belt, as shown in FIGS. 4C to 4E, the sensing mechanism 114 can be designed to locate at the buckling position and/or the surface of the belt contacting the user's skin. Therefore, it is known that the type of the sensing mechanism can be modified to conform to different kinds of users, for example, a baby or an aged person.

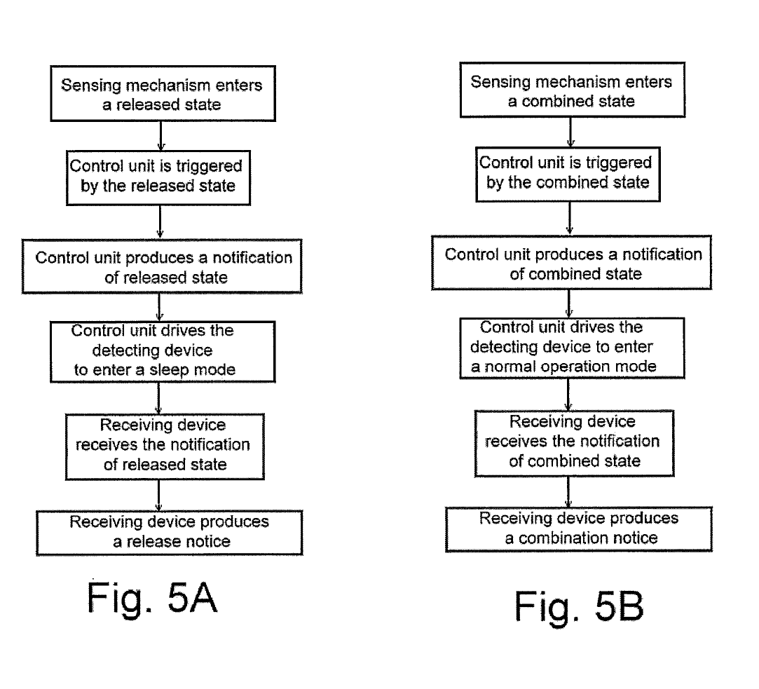

The notifying process of the sensing mechanism is shown in FIG. 5. The sensing mechanism is connected to the control unit so that the control unit can respond to a combined state and a released state of the sensing mechanism. As shown in FIG. 5A, when the sensing mechanism is released, the control unit is triggered to send out a notification of released state to the receiving device, and at the same time, the detecting device will get into a low-power consumption mode, such as a sleep mode. Then, after the receiving device receives the notification of released state, it will generate a release notice, for example, sound, image, light, vibration etc., for noticing the monitor that the detecting device is coming off the user. Therefore, the separation of the detecting device can be accurately detected, and of course, the false warning can be avoided.

Oppositely, as shown in FIG. 5B, a response also can be produced when the sensing mechanism is combined. For example, the combined state of the sensing mechanism may trigger the control unit to send out a notification of combined state to the receiving device, and after receiving the notification of combined state, the receiving device may produce a combination notice for informing the monitor. Therefore, if the combination notice is produced after the release notice, then the monitor can realize that the detecting device is reconstructed, no matter by the user himself or by other people. And, if the release notice is not followed by the combination notice, then the monitor can send a person to help the reconstruction of the detecting device. Here, opposite to the notification of released state which may simultaneously lower down the power consumption of the detecting device, the notification of combined state may also trigger the control unit to recover the detecting device back to a normal operation mode. Additionally, the combined state of the sensing mechanism can trigger the control unit to start the operation of the detecting device, so that, at the beginning of the measurement, the monitoring can be automatically started.

Consequently, the warning process of the sensing mechanism can be altered according to all kinds of demands only if the release notice can be differentiated from the warning notice.

In addition, except the warning, release and combination notices, the non-invasive vital sign monitoring apparatus also can employ other kinds of notices, for example, notices for notifying power condition, connection condition of detecting element, and/or operation condition, so as to provide more information to the monitor. And, the noticing method also can have different choices, such as, using different colors of the light, utilizing the light sparkle, and employing sound and/or image variation.

According to the above descriptions of the detecting device, the receiving device may have a corresponding design.

The receiving device 102 can include a displaying element 104 for showing the physiological information and status related to the user and the conditions of the detecting device, and an operation interface 106 for facilitating the monitor's operation. Here, particularly, through the operation interface 106, the monitor may remotely request the detecting device to transmit back the needed information, such as, the physiological information related to the user, information regarding the environment, and the conditions of the detecting device.

The contents of the information transmitted back by the detecting device owing to the request from the monitor can have different choices.

In one embodiment, it is designed that the detecting device only transmits a physiological information summary during the monitoring period, and as the monitor requires more detailed physiological information, he or she can request the detecting device to transmit back the detailed physiological information through the operation interface. Therefore, after the detecting device receives the requesting message, it will transmit back the requested information. More particularly, according to the present invention, it also can be designed that only partial detecting elements of the detecting device operate at a normal operation mode, and the originally un-operated detecting elements are only initiated as the monitor submits the request. For example, the detecting device can include a sound sensing element (as indicated by 122 in FIG. 1), such as a microphone, which is operated only when the requesting message is received, so that the sound of the user or the environment around the user can be transmitted back to the receiving device as requested. That is, the monitor dose not need to know the sound during all the monitoring period, but if a warning situation happens, the monitor can initiate the sound sensing element to gather more information, for example, whether the baby is crying, or even the breath sound of the user.

In another embodiment, as described above, for saving power, the detecting device according to the present invention does not perform the periodic transmission on the condition that the physiological signals are normal, but if the monitor needs to know the instant information, he or she can send out a requesting message to the detecting device through the operation interface, so that the detecting device will immediately be triggered to transmit back the instant physiological information once receiving the requesting message. Therefore, the monitor still can have the instant information under the power saving condition.

Furthermore, through the operation interface, the monitor also can designate the contents of the transmitted information, for example, if the detecting device includes the sound detecting element, such as, microphone, then the transmitted information can contain the sound collected by the microphone, or if the detecting device includes the temperature detecting element (as indicated by 124 in FIG. 1), then the monitor can know the instant temperature information, such as, the body temperature or the environmental temperature.

Consequently, according to the design of the present invention, the instant information requested by the monitor can have various types without limitation.

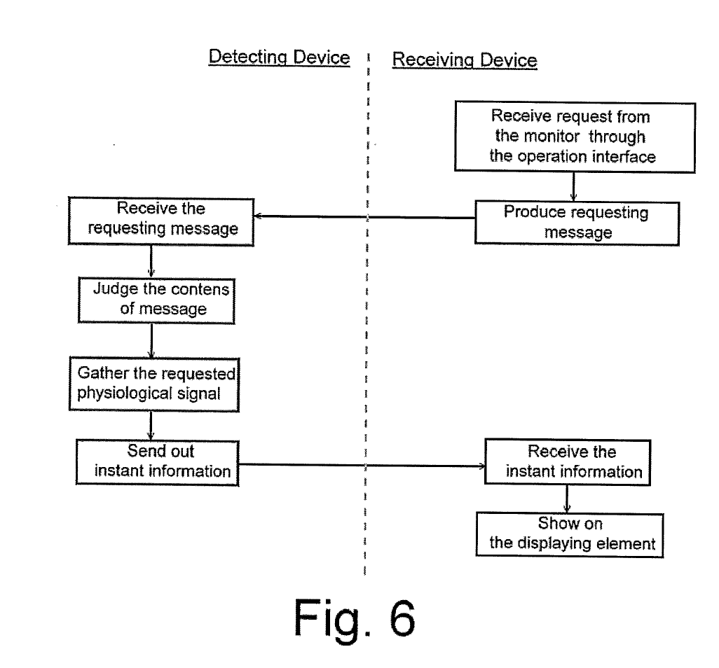

FIG. 6 is a flow chart showing the process that the receiving device actively requests the information transmission. As shown, firstly, the monitor selects the contents of the information through the operation interface. Then, after the detecting device receives the requesting message, it will gather the requested physiological signal and transmit it back to the receiving device. Finally, the physiological information is displayed on the displaying element after being received.

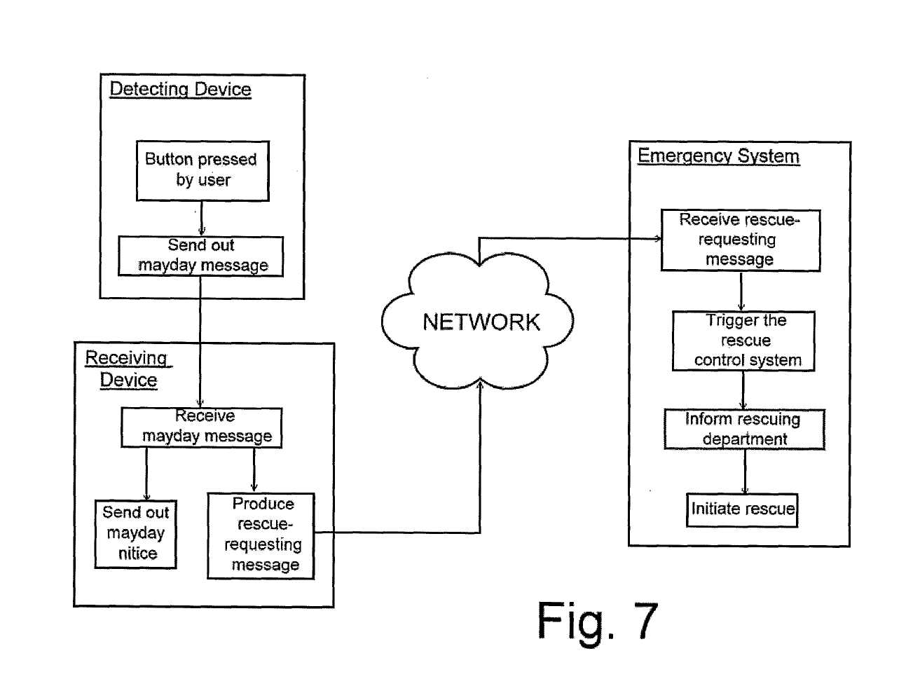

Opposite to the monitor utilizing the operation interface to request the detecting device to transmit information, the user to be monitored also can page the monitor aside the receiving device via the detecting device, as shown in FIG. 7. According thereto, the detecting device may further include a button (as indicated by 130 in FIG. 1), so that when the user has the need, for example, when he or she does not feel well or is in danger, the user can press the button to trigger the detecting device to send out a mayday message, and after the receiving device receives the mayday message, a mayday notice will be produced to notify the monitor. Simultaneously, except producing the mayday notice, the receiving device also can inform the emergency medical service system by sending out a rescue-requesting message for shortening the waiting time. This design is specifically benefit to the aged person who lives alone, so that, in addition to the auto warning process, he or she also can actively send out the mayday message through pressing the button. The security is enhanced.

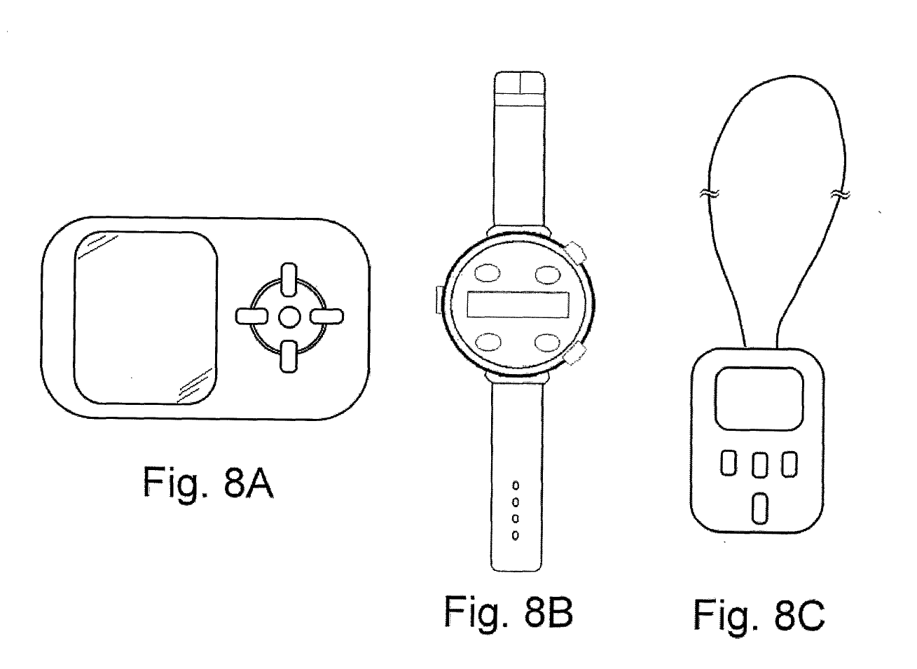

Then, please refer to FIGS. 8A to 8C, which show the possible examples of the receiving device according to the present invention. Since the receiving device is used by the monitor who might have the necessity to move, for example, a nurse moving in the hospital, it is better to implement the receiving device as a portable device, so that the monitor does not have to stay aside the receiving device. Therefore, as shown, the receiving device can be implemented to be an easy carried and operated device, for example, a hand-held device (FIG. 8A), a watch (FIG. 8B), or a neck-hanged device (FIG. 8C). Furthermore, for facilitating the operation, an assisting tool, such as, headset or touch pen, also can be provided.

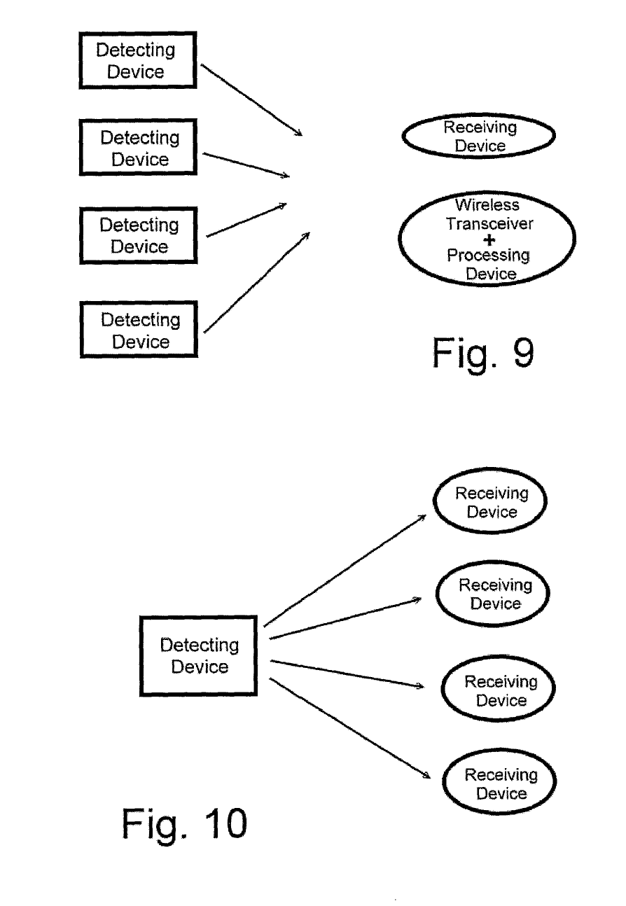

Besides, since the carer might have to take care of more than one person, such as the health nursing center in a geriatric community, or the monitoring center for baby caring, the receiving device can be implemented to be able to receive signals and messages from multiple detecting devices at the same time, as shown in FIG. 9. Therefore, the carer does not have to carry multiple receiving devices for monitoring multiple patients. And, more advantageously, the portable receiving device also can be implemented to be able to connect with the computer, so that the carer can continue the monitoring through the display of the computer.

Alternatively, except for being implemented as portable, the receiving device also can be implemented to be a wireless transceiver with a processing device, for example, a dongle with a personal computer, a PDA or a desktop. Through this embodiment, the application scope becomes wider. For instance, the transceiver can be installed on the computer in the nursing department for direct monitoring, so that the nurses on duty can in turn take the monitoring, or the transceiver can be combined with any portable computing device for increasing mobility and convenience.

Of course, according to the present invention, as shown in FIG. 10, the signals and messages sent by one detecting device also can be received by multiple receiving devices. This embodiment provides a more completed and secured nursing for the patients since the warning notice will be shown up on multiple receiving devices.

Preferably, in consideration of long-term monitoring, the receiving device may further include a memory component, so that the information transmitted by the detecting device can be stored for later doctor observation. Besides, the detecting device also can include a memory component, for example, to be a buffer before data transmission.

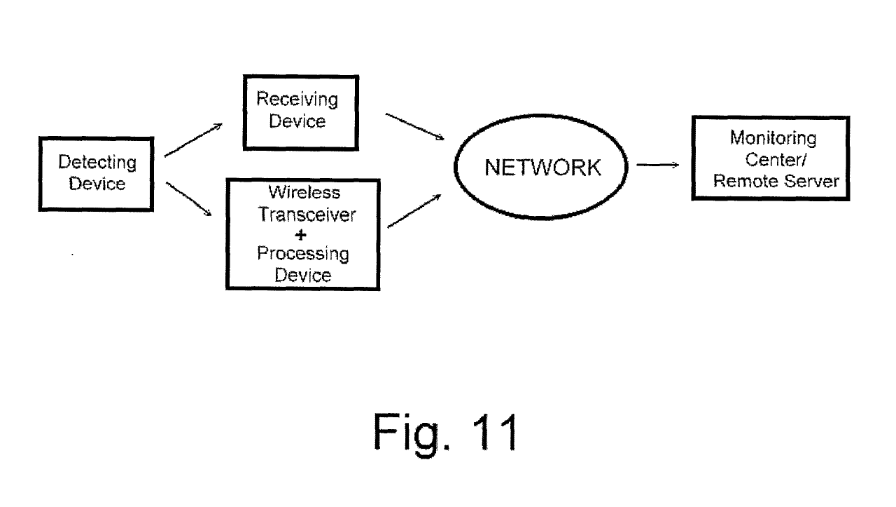

According to another aspect of the present invention, a non-invasive vital sign monitoring system is provided, as shown in FIG. 11. In this system, except the non-invasive vital sign monitoring apparatus, as described above, an administration console is also included, wherein the vital sign monitoring apparatus is connected with the administration console, as shown in FIG. 10, for example, through a network, wired or wireless, or through a direct connection, so that the administration console can instantly monitor the vital sign of the patient. Here, the administration console might be, but not limited, a monitoring center in a same building, a monitoring center at a remote place, or an emergency medical service system. Therefore, the signals and messages from the detecting device are received by the receiving device, and then the receiving device, as needed, sends out the message to the monitoring system for notifying, or more advantageously, the detecting device even can simultaneously inform the receiving device and the monitoring system. Preferably, if the detecting device equips GPS, then it will be easier to locate the patient. Besides, the monitoring person aside the administration console also can request the detecting device to transmit back the instant physiological information. Therefore, a two-directional nursing system can be completed.

Here, if the receiving device is communicated with the administration console through the network, then it can be, for example, the receiving device directly has a networking capability or the receiving device is connected to a computer to utilize the networking capability thereof.

In the aforesaid, the present invention provides a non-invasive vital sign monitoring apparatus which utilizes a non-invasive detecting element, which is advantageous of easy installation, to gather physiological signals. Moreover, the light weight device can be attached to the user through an attaching element, so that the user will not feel the loading and can have a great mobility. Furthermore, the attaching element is designed to have a sensing mechanism which can sense if the detecting device is coming off the human body and send out a notification, so that a false warning caused by device coming off can be avoided. Besides, except of monitoring, the receiving device can actively request the detecting device to transmit back information through an operation interface thereon, so that the receiving device and the detecting device can have a two-directional communication without limitation. In addition, as the receiving device is connected with an administration console, then an accurate, instant and convenient non-invasive vital sign monitoring system is achieved.

The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

1. A non-invasive vital sign monitoring apparatus, comprising:
a detecting device, attached to a first user through an attaching element, comprising:
a detecting unit, for gathering a physiological signal from the first user;
a sensing mechanism, mounted on the attaching element, having a combined state and a released state for corresponding to the attaching relationship thereof with the attaching element; and
a control unit, for controlling the operation of the detecting device, wherein the control unit triggers a warning message when the gathered physiological signal does not conform to a preset physiological condition, the control unit triggers a notification of released state when the sensing mechanism is under the released state, and in accordance with the states of the sensing mechanism, the control unit drives the detecting device to enter different operation modes; and
a receiving device, held by a second user, for wirelessly receiving the signals and messages from the detecting device, wherein
when receiving the warning message, the receiving device sends out a warning notice to show the second user a disconformity between the gathered physiological signal and the preset physiological condition; and
when receiving the notification of released state, the receiving device sends out a release notice to the second user.

2. The apparatus as claimed in claim 1, wherein the attaching element is implemented to be a winding object, an adhering patch or a hanging belt.

3. The apparatus as claimed in claim 1, wherein the physiological signal is a heartbeat signal and/or a pulse signal.

4. The apparatus as claimed in claim 1, wherein the detecting unit is an optical detecting unit comprising an optical emitting element and an optical receiving element, and the quantity of the optical emitting element and the optical receiving element are both changeable, and wherein the optical detecting unit is attached to the first user by clipping, surrounding or adhering, and at finger, ear, or forehead.

5. The apparatus as claimed in claim 1, further comprising a piezo-type detecting element, attached to the first user, for gathering another kind of physiological signal from the first user.

6. The apparatus as claimed in claim 5, wherein the piezo-type detecting element is used for detecting signals generated by skin surface vibration and/or skin expansion/contraction, and the piezo-type detecting element is attached to the first user by adhering, surrounding or binding, and at the chest or the neck.

7. The apparatus as claimed in claim 1, further comprising a non-invasive physiological electrode, attached to the first user, for gathering another kind of physiological signal from the first user.

8. The apparatus as claimed in claim 1, further comprising a movement sensing element.

9. The apparatus as claimed in claim 8, wherein the movement sensing element is located inside the detecting device, and the signals gathered by the movement sensing element are used to be a judging reference of other signals.

10. The apparatus as claimed in claim 1, further comprising a sound sensing element, for providing a sound output by the first user or by the environment around the first user to the second user.

11. The apparatus as claimed in claim 1, further comprising a temperature sensing element, for providing a body temperature information and/or an environment temperature information.

12. The apparatus as claimed in claim 1, wherein the combined state of the sensing mechanism triggers the control unit to send out a notification of combined state, and the sensing mechanism is located between the attaching element and the detecting device, and/or between the attaching element and the first user.

13. The apparatus as claimed in claim 1, wherein the detecting device further comprises a button, for being used by the first user to trigger a mayday message, and then the receiving device, after receiving the mayday message, sends out a mayday notice and simultaneously sends out a rescue-requesting message to an emergency system.

14. The apparatus as claimed in claim 1, wherein the receiving device further comprises a displaying element and an operation interface, so that the second user is capable of utilizing the operation interface to send out a requesting message for requesting the detecting device to transmit back a particular physiological signal of the first user.

15. The apparatus as claimed in claim 1, wherein the notices sent by the receiving device are implemented as sound, light, image and/or vibration.

16. The apparatus as claimed in claim 1, wherein the signals and messages sent by the detecting device are received by multiple receiving devices.

17. The apparatus as claimed in claim 1, wherein the receiving device receives signals and messages from multiple detecting devices.

18. A non-invasive vital sign monitoring apparatus, comprising:
a detecting device, attached to a first user through an attaching element for gathering physiological signals from the first user, comprising:
a sensing mechanism, mounted on the attaching element, having a combined state and a released state for corresponding to an attaching relationship thereof with the attaching element,
wherein according to the states of the sensing mechanism, the detecting device are changed to enter different operation modes; and
a receiving device, held by a second user, comprising:
an operation interface, which is utilized by the second user to send out a requesting message for requesting the detecting device to transmit back a particular physiological signal.

19. The apparatus as claim in claim 18, wherein the different operation modes have different power supplying conditions.

20. The apparatus as claim in claim 18, wherein the released state of the sensing mechanism triggers the detecting device to send out a notification of released state, and then, the receiving device, after receiving the notification of released state, sends out a release notice, and wherein the combined state of the sensing mechanism triggers the detecting device to send out a notification of combined state.

21. The apparatus as claim in claim 18, wherein the detecting device sends out a warning message when the gathered physiological signal does not conform to a preset physiological condition, and the receiving device, as receiving the warning message, sends out a warning notice.

22. A non-invasive method for monitoring physiological signals, comprising steps of:
a) providing a detecting device with an attaching element and a receiving device;
b) providing a sensing mechanism, having a combined state and a release state, on the detecting device;
c) attaching the detecting device on a user through the attaching element;
d) gathering a physiological signal;
e) monitoring the states of the sensing mechanism;
f) sending out a notification of released state to the receiving device as the detecting device detects the released state; and
g) sending out a release notice as the receiving device receives the notification of released state for showing the release state.

23. The method as claimed in claim 22, wherein the sensing mechanism is located between the attaching element and the detecting device and/or between the attaching element and the user.

24. The method as claimed in claim 22, further comprising the steps of:
h) as the combined state is detected, sending out a notification of combined state by the detecting device to the receiving device; and
i) sending out a combination notice by the receiving device after receiving the notification of combined state.

25. A non-invasive method for monitoring physiological signals, comprising steps of:
a) providing a detecting device with an attaching element and a receiving device;
b) providing an operation interface on the receiving device;
c) attaching the detecting device on a user through the attaching element;
d) gathering a physiological signal;
e) sending out a requesting message through the operation interface, so as to request the detecting device to transmit a particular physiological signal; and
f) transmitting the requested physiological signal to the receiving device after the detecting device receives the requesting message.

26. The method as claimed in claim 25, wherein the physiological signal is at least one of a group consisting of: a heart beat/pulse signal, a sound signal, a temperature signal and a movement signal.

27. A non-invasive vital sign monitoring system, comprising:
at least an non-invasive vital sign monitoring apparatus comprising:
a detecting device, attached to a user through an attaching element, comprising:
a detecting unit, for gathering a physiological signal from the first user;
a sensing mechanism, mounted on the attaching element, having a combined state and a released state for corresponding to an attaching relationship thereof with the attaching element; and
a control unit, for controlling the operation of the detecting device, wherein the control unit triggers a warning message when the gathered physiological signal does not conform to a preset physiological condition, the control unit triggers a notification of released state when the sensing mechanism is under the released state, and the control unit drives the detecting device to enter different operation modes in accordance with the states of the sensing mechanism; and
a receiving device for wirelessly receiving the physiological signals and messages from the detecting device; and
at least an administration console, wherein
when receiving the warning message, the receiving device sends out a warning notice to the administration console for showing a disconformity between the gathered signals and the preset physiological condition; and
when receiving the notification of released state, the receiving device sends out a release notice to the administration console for showing the attaching situation of the detecting device.

28. The system as claimed in claim 27, wherein the vital sign monitoring apparatus is connected with the administration console through a network system, which is a wired or wireless network system.

29. The system as claimed in claim 27, wherein the administration console sends out a requesting message for requesting a physiological signal from the detecting device.

30. The system as claimed in claim 29, wherein the requesting message is transmitted to the receiving device and then to the detecting device, and the detecting device transmits back the physiological signal after receiving the requesting message.
